(19) United States
(12) Patent Application Publication
(10) Pub. No.: US 2021/0270767 A1
(43) Pub. Date: Sep. 2, 2021

(54) METHOD FOR OPERATING A SENSOR FOR DETECTING AT LEAST A PORTION OF A MEASUREMENT GAS COMPONENT HAVING BOUND OXYGEN IN A MEASUREMENT GAS

(71) Applicant: Robert Bosch GmbH, Stuttgart (DE)

(72) Inventors: Andy Schroeder, Daettwil (CH); Dirk Daecke, Stuttgart (DE); Michael Heise, Radebeul (DE); Michael Fiedler, Waiblingen (DE); Mustafa Guel, Backnang (DE); Peter Oechtering, Karlsruhe (DE); Matthias Singer, Esslingen (DE)

(21) Appl. No.: 17/255,627

(22) PCT Filed: Jun. 26, 2019
(86) PCT No.: PCT/EP2019/066943

§ 371 Date: Dec. 23, 2020

(30) Foreign Application Priority Data
Jul. 2, 2018 (DE) 10 2018 210 846.3

(51) Int. Cl. G01N 27/407; G01N 27/41; G01M 15/10

(52) U.S. Cl. CPC G01N 27/4071; G01M 15/102; G01N 27/41

(57) ABSTRACT

A method for operating a sensor for detecting at least a portion of a measured-gas component having bound oxygen in a measured gas in an exhaust gas of an internal combustion engine. The sensor encompasses a sensor element including: a first pump cell; a reference cell; and a second pump cell. An electronic control device is connected to the sensor element; the first pump cell is connected using an electrically conductive connection to a first separate terminal of the device; the second pump cell is connected by an electrically conductive connection to a second separate terminal of the device; a measuring resistor is provided in the connection that connects the second pump cell to the second separate terminal; a current excitation and/or voltage excitation of the second pump cell is carried out using the control device to generate a measured signal at the measuring resistor.

### BACKGROUND INFORMATION

In the related art, there exists a number of methods and sensors for detecting at least a portion of the measured-gas component having bound oxygen in a gas mixture, in particular in an exhaust gas of an internal combustion engine, by identifying an oxygen portion that is generated by reduction of the measured-gas component having the bound oxygen.

Sensors for detecting at least a portion of the measured-gas component having bound oxygen in a gas mixture, which are also referred to in abbreviated or simplified fashion as “NOx sensors” or “nitrogen oxide sensors,” are described, for example, in Reif, K., Deitsche, K.-H., et al., Kraftfahrtechnisches Handbuch [Automotive handbook], Springer Vieweg, Wiesbaden, 2014, pp. 1338-1347.

The nitrogen oxide sensors (═NOₓ sensors) that are used nowadays in automotive engineering operate in accordance with the limit current principle, analogously to oxygen sensors, for example lambda sensors. A nitrogen oxide sensor of this kind encompasses a Nernst concentration cell (also called a “reference cell”), a modified oxygen-pump cell, and a further modified oxygen-pump cell (the so-called “NOₓ cell”). An external pump electrode exposed to the exhaust gas, and an internal pump electrode in a first cavity that is separated from the exhaust gas by a diffusion barrier, constitute the oxygen-pump cell. The Nernst electrode is also located in the first cavity, and the reference electrode is located in a reference gas space, together constituting the Nernst cell or reference cell. The NOₓ cell encompasses an NOₓ pump electrode and a counter-electrode. The NOₓ pump electrode is located in a second cavity that is connected to the first internal cavity and is separated therefrom by a diffusion barrier. The counter-electrode is located in the reference gas space. All the electrodes in the first and the second cavity have a common return lead.

Upon operation of the nitrogen oxide sensor, oxygen is removed from the so-called “02 cell” out of the first cavity, which is connected via a diffusion barrier to the exhaust gas. The pump current resulting therefrom is then proportional to the oxygen content of the ambient air in the measured-gas or exhaust-gas flow. The nitrogen oxides are pumped off in the NOₓ cell. The nitrogen oxide (NOₓ) in the atmosphere present in the second cavity is reduced or decreased by application of a constant pump voltage. The oxygen generated by the reduction or decrease of the measured-gas component in the second cavity, which preferably results from the reduction of the nitrogen oxide (NOₓ), is pumped off into a reference gas space. The pump voltage that is applied against the resistance of the NOₓ cell and the concentration of the nitrogen oxide (NOₓ) or oxygen thus results in a pump current that is proportional to the concentration of nitrogen oxide (NOₓ) or oxygen, and represents the measured NOₓ signal.

The pump current I_{P2} that results in this context is thus an indication of the NOₓ concentration of the ambient air in the measured-gas or exhaust-gas flow. With this assemblage, it is important that the nitrogen oxides not also be pumped off at the oxygen cell, since otherwise a signal would no longer be measurable at the NOₓ cell. This is achieved by gold doping of the O2 cell. In addition, the O2 cell must be operated only at low pump voltages, since otherwise NOₓ molecules would become dissociated again.

Despite the advantages of the conventional sensors and of the methods for operation thereof, there is still potential for improving them. The temperature of the sensor element is controlled by pulse width modulation (PWM) of the heater power supply (voltage, current). The PWM voltage is tapped off via a field effect transistor (FET) directly from the supply voltage (12 V) of the sensor control unit (SCU). The result is that during the PWM “on” phase, the SCU supply voltage of the system is applied to the heating coil of the sensor element in the sensor probe. The current of the measured NOₓ signal is very low, for example 4.5 ρA for 1500 ppm NOₓ, and is thus also extremely sensitive to disruptions and incoupling. Because of the physical proximity of the heating coil to the NOₓ cell, a current is impressed onto the IP2 lead/cell during the “on” phase of the PWM signal due to capacitive coupling and leakage currents. An offset with respect to the actual NOₓ value is measurable as a result of this interference. Environmental authority regulations require continuous and reliable diagnosis of lead breakages in the IP2 lead.

### SUMMARY

In accordance with an example embodiment of the present invention, a method for operating a sensor for detecting at least a portion of a measured-gas component having bound oxygen in a measured gas is provided, which method may at least largely avoid the disadvantages of conventional methods for operating such sensors and permits reliable and continuous diagnosis at intervals of at least 500 ms without influencing or interfering with the measured NOₓ values.

In a method according to an example embodiment of the present invention for operating a sensor for detecting at least a portion of a measured-gas component having bound oxygen in a measured gas, in particular in an exhaust gas of an internal combustion engine, the sensor encompassing a sensor element, the sensor element has: a first pump cell that has an external pump electrode and an internal pump electrode and adjoins a first cavity that is in communication with the measured gas; a reference cell that has a Nernst electrode and a reference electrode and adjoins a reference gas space; and a second pump cell that has a pump electrode and a counter-electrode and adjoins a second cavity, an electronic control device, which possesses at least a first separate terminal for the first pump cell and a second separate terminal for the second pump cell, is connected to the sensor element; the first pump cell being connected by way of an electrically conductive connection to the first separate terminal; the second pump cell being connected by way of an electrically conductive connection to the second separate terminal; a measuring resistor being provided in the electrically conductive connection that connects the second pump cell to the second separate terminal; a current excitation and/or voltage excitation of the second pump cell being carried out by way of the control device in order to generate a measured signal at the measuring resistor.

The result of the current excitation and/or voltage excitation of the second pump cell is to generate at the measuring resistor an evaluatable measured signal that permits a distinction between an open or a closed circuit. In other words, a signal IP2 is generated by an external excitation. An open circuit on the IP2 lead of the NOₓ cell can thus be reliably recognized even during measurement operation. An open IP2 lead can be detected even in operating states in which the IP2 current is equal to (approximately) zero, for example at 0 ppm NOₓ.

In a refinement of the present invention, a predetermined electrical voltage is applied to the second pump cell; a voltage excitation of the second pump cell being carried out; the voltage excitation encompassing a modification of the predetermined electrical voltage for a predetermined time. Excitation of the voltage with respect to the normal potential results, in the context of a closed lead and with a gas mixture, even at 0% O2, H2O≥1%, and NOₓ=0 ppm, in a measurable current flow. If the IP2 lead is severed, no further current can flow, not even in a context of a time-related excitation by way of a pulse. This serves as an indication of an open circuit. What is important here is not the duration of the pulse but the change in voltage.

In a refinement of the present invention, the predetermined electrical voltage is raised for the predetermined time. The measurable current flow also rises as a result.

In a refinement of the present invention, the electrically conductive connection that connects the second pump cell to the second separate terminal is identified as intact if the measured signal has a value not equal to zero for the predetermined time, and is identified as defective if the measured signal has a value of zero for the predetermined time. It is thereby possible to distinguish unequivocally between an open and a closed circuit.

In a refinement of the present invention, a predetermined electrical voltage is applied to the second pump cell; a voltage excitation of the second pump cell being carried out; the predetermined electrical voltage being raised for a first predetermined time, and the predetermined electrical voltage being lowered for a second predetermined time; an integral of the applied electrical voltage having a value of zero for the first predetermined time and the second predetermined time. With the NOₓ sensor in the measurement state, the voltage U_{P2} at the NOₓ cell is therefore briefly raised by the hardware by an amount equal to a specific potential, and then minimized in the opposite potential direction for the same time. It is immaterial whether the positive voltage pulse or the negative voltage pulse is carried out first. Excitation of the voltage with respect to the normal potential causes a measurable current flow in the context of a closed lead with a gas mixture, including at 0% O2, H2O≥1%, and NOₓ=0 ppm. If the IP2 lead is severed, a current can no longer flow even in the context of a time-related excitation by a pulse. This is considered an indication of an open circuit.

In a refinement of the present invention, the electrically conductive connection that connects the second pump cell to the second separate terminal is identified as intact if the measured signal has a value not equal to zero for the first predetermined time and the second predetermined time, and is identified as defective if the measured signal has a value of zero for the first predetermined time and the second predetermined time. The integral of the voltage amplitude over both pulses must equal zero over the time of both pulses, i.e., must be offset-free, in order to indicate a closed circuit. Otherwise an indication of an open circuit exists.

In a refinement of the present invention, a predetermined electrical current is impressed into the second pump cell; a current excitation of the second pump cell being carried out; the predetermined electrical current being raised for a first predetermined time, and the predetermined electrical current being lowered for a second predetermined time; the first predetermined time and the second predetermined time being identical in length. An alternative to the voltage pulse is a current pulse on the IP2 lead. With the NOₓ sensor in the measurement state, a current pulse is briefly raised by the hardware by an amount equal to a specific value, and then minimized for the same time in the opposite direction. It is immaterial whether the positive current pulse or the negative current pulse is carried out first. Impression of a current pulse (pump current) causes a voltage excursion on the IP2 lead to become measurable. That excursion behaves differently with a closed circuit than with an open circuit. This can be taken as a distinguishing feature for detection of an open circuit on the IP2 lead.

In a refinement of the present invention, the second electrically conductive connection is identified as intact if an electrical voltage applied to the second pump cell falls below a threshold value for the first predetermined time and for the second predetermined time, and is identified as defective if an electrical voltage applied to the second pump cell exceeds a threshold value for the first predetermined time and for the second predetermined time. The integral of the current over both pulses must be equal to zero over the time of both pulses in order not to bring about any imbalance in the NOₓ cell due to one-sided pumping up or pumping down. Impression of a current pulse (pump current) causes a voltage excursion on the IP2 lead to become measurable. That excursion behaves differently with a closed circuit than with an open circuit. For example, the voltage excursion is less with a closed circuit than with an open circuit. This can be taken as a distinguishing feature for detection of an open circuit on the IP2 lead.

In a refinement of the present invention, a predetermined electrical voltage is applied to the second pump cell; a voltage excitation of the second pump cell is carried out; the voltage excitation encompassing a periodic modification of the predetermined electrical voltage. The application of a voltage excitation on the IP2 lead causes a definite current change at the NOₓ cell. The amplitude of the modulated signal can be very low if the frequency is high enough. The frequency on the IP2 lead does not obligatorily need to be excited over the entire time span. Application of the higher-frequency voltage modification in the region around almost 0 ppm NOₓ is sufficient for electrical diagnosis. If a current excited by the frequency is measurable on the raw signal, in particular at 0% O2, H2O 1%, and NOₓ=0 ppm, the IP2 lead is OK. If no current is measurable on the raw signal, an open circuit must then be present at the NOₓ cell lead. The amplitude shape is then immaterial. The greater the change over time d/dt (U)t in the voltage, however, the more pronounced the change in current.

In a refinement of the present invention, the period length is less than an electrochemical time constant of the second pump cell. Application of a voltage excitation on the IP2 lead whose period length is much less than the (electrochemical) time constant of the NOₓ cell causes a definite current change at the NOₓ cell. The amplitude of the modulated signal can be very low if the frequency is high enough. The frequency of the voltage change should be selected so that the NOₓ output signal is not interfered with. The frequency on the IP2 lead does not obligatorily need to be excited over the entire time span. Application of the higher-frequency voltage modification in the region around almost 0 ppm NOₓ is sufficient for electrical diagnosis. If a current excited by the frequency is measurable on the raw signal, in particular at 0% O2, H2O≥1%, and NOₓ=0 ppm, the IP2 lead is OK. If no current is measurable on the raw signal, an open circuit must then be present at the NOₓ cell lead. The amplitude shape is then immaterial. The greater the change over time d/dt (U)t in the voltage, however, the more pronounced the change in current.

In a refinement of the present invention, the measured signal is filtered by way of a low-pass filter. Typically, the raw signal is filtered with a low-pass filter and transferred via the interface of the sensor control device to the engine control device. As a result of the frequency incoupling, the change in the current on the raw signal of the IP2 current becomes visible and can be used for electrical diagnosis. The NOₓ values that result from the IP2 current are filtered with a low-pass filter before they are sent onto the CAN. This higher-frequency current change is then no longer visible on the filtered signal.

In a refinement of the present invention, the predetermined electrical voltage is modified at a frequency that is greater than the bandwidth of the low-pass filter. Usefully, however, the excitation frequency should also lie within the bandwidth of the analog-to-digital converter (ADC). As a result of the frequency incoupling, the change in the current on the raw signal of the IP2 current becomes visible and can be used for electrical diagnosis. The NOₓ values that result from the IP2 current are filtered with a low-pass filter before they are sent onto the CAN. This higher-frequency current change is then no longer visible on the filtered signal.

In a refinement of the present invention, the electrically conductive connection that connects the second pump cell to the second separate terminal is identified as intact if the (unfiltered) measured signal exhibits a periodic change, and is identified as defective if the measured signal exhibits no periodic change. If a current excited by the frequency is measurable on the raw signal, in particular at 0% O2, H2O≥1%, and NOₓ=0 ppm, the IP2 lead is OK. If no current is measurable on the raw signal, an open circuit must then be present at the NOₓ cell lead. The amplitude shape is then immaterial. The greater the change over time d/dt (U)t in the voltage, however, the more pronounced the change in current.

In accordance with an example embodiment of the present invention, a computer program is also provided that is configured to carry out each step of the method according to the present invention.

Additionally, in accordance with an example embodiment of the present invention, an electronic storage medium is provided on which a computer program for carrying out the method according to the present invention is stored.

The present invention furthermore encompasses an electronic control device that encompasses the electronic storage medium according to the present invention having the aforesaid computer program for carrying out the method according to the present invention.

The present invention also relates to a sensor for detecting at least a portion of a measured-gas component having bound oxygen in a measured gas, in particular in an exhaust gas of an internal combustion engine, encompassing a sensor element. In accordance with an example embodiment of the present invention, the sensor element includes: a first pump cell that has an external pump electrode and an internal pump electrode and adjoins a first cavity that is communication with the measured gas; a reference cell that has a Nernst electrode and a reference electrode and adjoins a reference gas space; and a second pump cell that has a pump electrode and a counter-electrode and adjoins a second cavity; the sensor furthermore having an electronic control device according to the present invention.

A “solid electrolyte” is to be understood in the context of the present invention as a body or object having electrolytic properties, i.e., having ion-conducting properties. It can be, in particular, a ceramic solid electrolyte. This also encompasses the raw material of a solid electrolyte and therefore the embodiment as a so-called green compact or brown compact, which becomes a solid electrolyte only after sintering. The solid electrolyte can be embodied in particular as a solid-electrolyte layer or from several solid-electrolyte layers. A “layer” is to be understood in the context of the present invention as a uniform mass which has a planar extent of a certain height and which is located above, below, or between other elements.

An “electrode” is to be understood generally in the context of the present invention as an element that is capable of contacting the solid electrolyte in such a way that a current through the solid electrolyte and the electrode can be maintained. The electrode can correspondingly encompass an element at which ions can be incorporated into the solid electrolyte and/or removed from the solid electrolyte. The electrodes typically encompass a noble-metal electrode, which for example can be applied as a metal-ceramic electrode on the solid electrolyte or can be connected in another manner to the solid electrolyte. Typical electrode materials are platinum cermet electrodes. In general, however, other noble metals, for example gold or palladium, are also usable.

A “heating element” is to be understood in the context of the present invention as an element that serves to heat the solid electrolyte and the electrodes at least to their functional temperature and preferably to their operating temperature. The functional temperature is the temperature above which the solid electrode becomes conductive for ions, and is equal to approximately 350° C. This is to be distinguished from the operating temperature, which is the temperature at which the sensor element is usually operated and which is higher than the functional temperature. The operating temperature can be, for example, from 700° C. to 950° C. The heating element can encompass a heating region and at least one supply lead trace. A “heating region” is to be understood in the context of the present invention as that region of the heating element which overlaps with the electrode in the layered structure in a direction perpendicular to the surface of the sensor element. The heating region usually heats up during operation more intensely than does the supply lead trace, so that they are distinguishable. The different heating can be implemented, for example, by the fact that the heating region has a higher electrical resistance than the supply lead trace. The heating region and/or the supply lead are embodied, for example, as an electrical resistance trace, and heat up due to application of an electrical voltage. The heating element can be manufactured, for example, from a platinum cermet. The present invention is directly detectable by way of a shortened waiting time until operational readiness is reached after startup of the sensor. The respective potentials can be measured at the supply leads.

A “voltage excitation” of the second pump cell is to be understood in the context of the present invention as the application of an electrical voltage not equal to 0 V to the second pump cell or to the electrical lead to the NOₓ counter-electrode. The voltage excitation generates a current flow through the pump cell and the electrical lead to the NOₓ counter-electrode. This current flow produces a detectable measured signal at a measuring resistor in that lead.

A “current excitation” of the second pump cell is to be understood in the context of the present invention as the impression of an electrical current not equal to 0 A into the second pump cell or the electrical lead to the NOₓ counter-electrode. Impression of a current pulse (pump current) brings about a voltage excursion on the electrical lead to the NOₓ counter-electrode, which excursion is measurable at a measuring resistor in that lead. That excursion behaves differently with a closed circuit than with an open circuit.

A “low-pass” is to be understood in the context of the present invention as a filter that allows the passage of low-frequency signal components below its limit frequency, while high-frequency signal components are damped. The purpose of the low-pass filter is to sum the signal course of the difference signal over the time interval of the interference. If the difference signal is small, the compensation current signal will go toward zero. A simple low-pass implementation can be achieved, for instance, with a leaky integrator. In an alternative implementation, the properties of the low-pass can be modified depending on the magnitude of the input signal.

The present invention can be easily and effectively detected by monitoring the electrical signals on the NOₓ cell lead. If, in particular, a voltage pulse or current pulse is measured with an oscilloscope on the lead between the sensor and control device during normal measurement operation, the circuits and methods described in connection with the present invention are then being used.

### BRIEF DESCRIPTION OF THE DRAWINGS

Further optional details and features of the present invention are described below in the context of preferred exemplifying embodiments that are depicted in the Figures.

FIG. 1 shows schematically shows the construction of a sensor according to an example embodiment of the present invention.

FIG. 2 shows part of a sensor with part of a control device connected thereto, in accordance with an example embodiment of the present invention.

FIG. 3 shows a first example of a time course of electrical voltages and a measured signal in the context of the sensor, in accordance with the present invention.

FIG. 4 shows a second example of a time course of electrical voltages and a measured signal in the context of the sensor, in accordance with the present invention.

FIG. 5 shows a third example of a time course of electrical voltages, electrical currents, and a measured signal in the context of the sensor, in accordance with the present invention.

### DETAILED DESCRIPTION OF EXAMPLE EMBODIMENTS

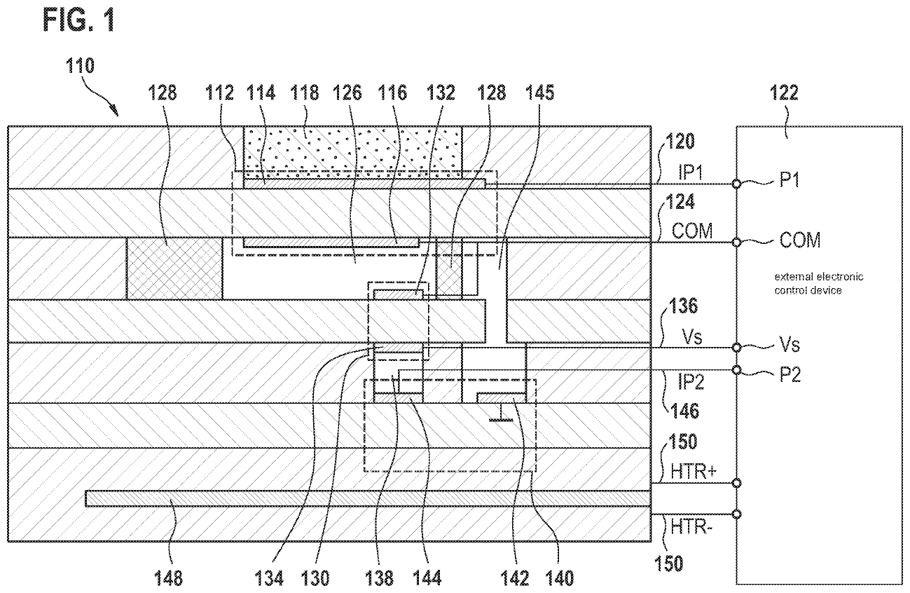

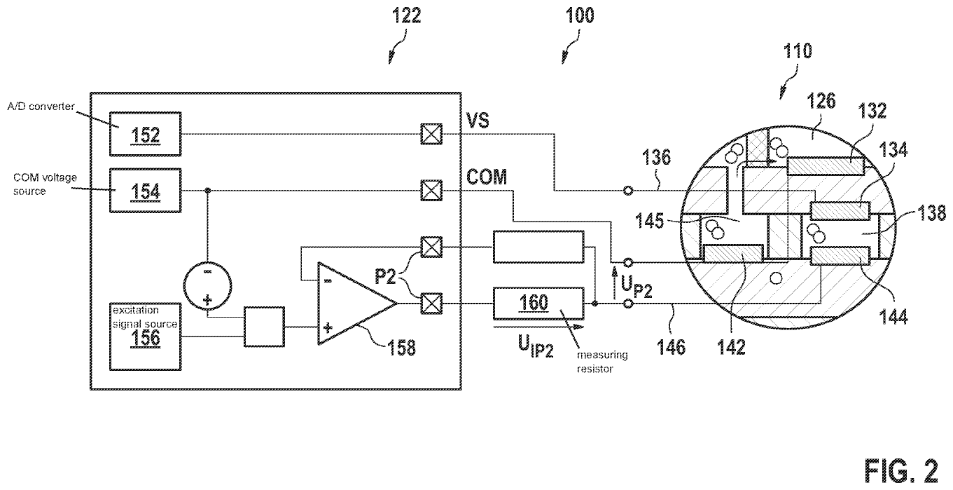

FIG. 1 schematically shows a construction of a sensor 100 according to the present invention which is particularly suitable for carrying out the method according to the present invention.

Sensor 100, which is configured to detect at least a portion of a measured-gas component having bound oxygen, hereinafter referred to by way of example as nitrogen oxide (NOₓ), in a gas mixture, by way of example an exhaust gas of an internal combustion engine, encompasses for that purpose a sensor element 110 and a first pump cell 112 that is embodied between an external pump electrode 114 and an internal pump electrode 116. External pump electrode 114, which is separated by way of a porous aluminum oxide layer 118 from the environment of sensor 100, possesses a first electrically conductive connection 120 by way of which a first pump current I_{P1} can be generated in first pump cell 112. First electrically conductive connection 120 is connected for that purpose to a first terminal P1 of an external electronic control device 122. In order to obtain a complete circuit, internal pump electrode 116 likewise possesses a second electrically conductive connection 124 that leads to a common terminal COM of external electronic device 122. First pump cell 112 adjoins a first cavity 126 that is located in the interior of sensor element 110 and is in communication with the measured gas. By generation of the first pump current I_{P1} in first pump cell 112, a first portion of oxygen ions which are formed from molecular oxygen from the gas mixture can be transported between first cavity 126 and the environment of sensor 100. Two diffusion barriers 128 are present in the entry passage from the environment into first cavity 126.

Sensor element 110 furthermore has an electrical reference cell 130 that has a Nernst electrode 132 and a reference electrode 134. While Nernst electrode 132 connects via second electrically conductive connection 124, together with internal pump electrode 116, to common terminal COM, reference electrode 134 has a separate third electrically conductive connection 136 to a supply voltage Vs that furnishes the required supply voltage Vs via a terminal Vs of external electronic control device 122. Reference cell 130 adjoins a reference gas space 138. A second portion of the oxygen ions from first cavity 126 and/or from the environment of sensor 100 is transported into reference gas space 138 by application of a reference pump current between terminal Vs and common terminal COM. The value for the reference pump current is adjusted in this context in such a way that a specified portion of the oxygen ions becomes established in reference gas space 138. Preferably, the value for the first pump current I_{P1} is also adjusted in this context in such a way that a specified ratio is produced between the first portion of the oxygen ions in first cavity 126 and the second portion of the oxygen atoms in reference gas space 138.

The measured-gas component, nitrogen oxide (NOₓ), which has the bound oxygen and is further contained in the gas mixture, travels, in particular by diffusion and in largely uninfluenced fashion, into a second pump cell 140 of sensor 110 which can also be referred to as an “NOₓ pump cell.” Second pump cell 140 has an NOₓ pump electrode 142 and an NOₓ counter-electrode 144, and adjoins a second cavity 145 in the interior of sensor element 110. Second cavity 145 is separated from first cavity 126 by one of diffusion barriers 128. At least one of the two electrodes (NOₓ pump electrode 142 and/or NOₓ counter-electrode 144) is configured in such a way that upon application of a voltage, further molecular oxygen can be generated by catalysis from the NOₓ measured-gas component and is formed in second pump cell 140.

Whereas NOₓ pump electrode 142 has an electrically conductive connection that leads to common terminal COM, NOₓ counter-electrode 144 has a fourth electrically conductive connection 146 by way of which a second pump current Iₚ₂ can be applied to second pump cell 140. Fourth electrically conductive connection 146 is connected for that purpose to a second terminal P2 of external electronic control device 122. Upon application of a second pump current I_{P2} to second pump cell 140, a portion of further oxygen ions that have been formed from the further molecular oxygen is transported into reference gas space 138.

Sensor element 110 furthermore possesses a heating element 148 that has a heating lead 150 having leads HTR+ and HTR− by way of which a heating current can be introduced into heating element 148 which, by generating a heat output, can bring sensor element 110 to the desired temperature.

Control device 122 has an analog-digital converter 152 that is connected to terminal Vs for the supply voltage. Control device 122 furthermore has a COM voltage source 154 that is connected to common terminal COM. Control device 122 furthermore has an excitation signal source 156 that is connected to the positive pole of an operational amplifier 158. In the exemplifying embodiment shown, operational amplifier 158 is a voltage follower. COM voltage source 154 is also connected to the positive pole of operational amplifier 158. Operational amplifier 158 is in turn connected to second terminal P2. A measuring resistor 160 is disposed in fourth electrical lead 146 between second terminal P2 and counter-electrode 144.

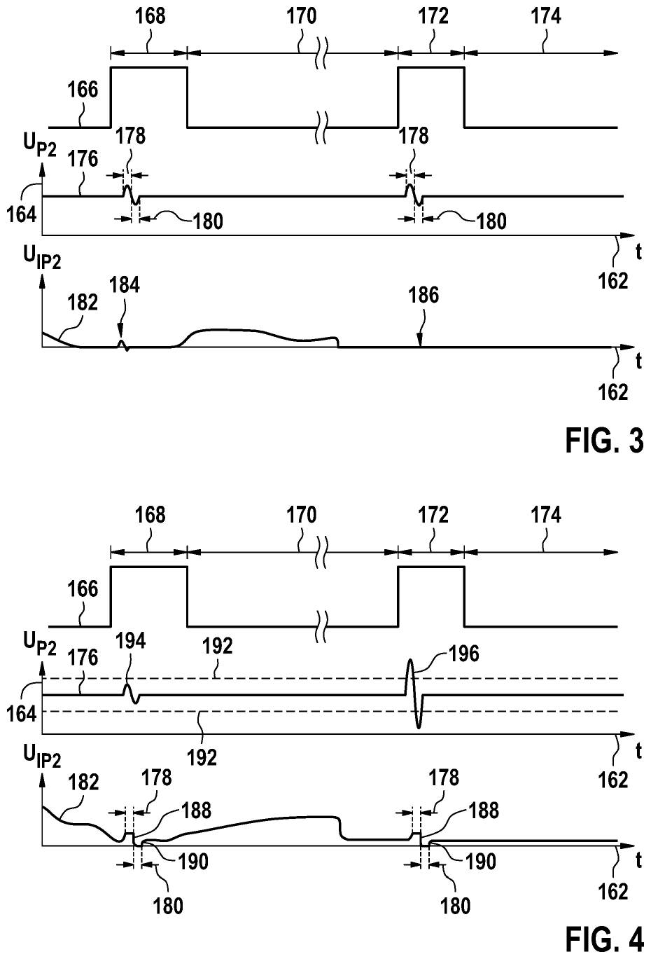

FIG. 3 shows a first example of a time course of electrical voltages and a measured signal in sensor 100. Time is plotted on X axis 162. Listed from top to bottom, the heating voltage of heating element 148, the electrical voltage U_{P2} applied to second pump cell 140, and the voltage drop U_{IP2} at measuring resistor 160 are plotted on Y axis 164. The voltage drop U_{IP2} at measuring resistor 160 represents the measured signal. The temperature of sensor element 110 is controlled by pulse width modulation (PWM) of the heater power supply (voltage or current). A curve 166 that represents the heating voltage of heating element 148 correspondingly shows at least a first phase or a first time span 168 in which an electrical voltage is applied to heating element 148 and heating element 148 is thus switched on, and a second phase or a second time span 170 in which no electrical voltage is applied to heating element 148 and heating element 148 is thus switched off. Second time span 170 follows first time span 168. A sum of first time span 168 and second time span 170 is, for example, 500 ms. In the exemplifying embodiment shown, curve 166 furthermore exhibits a third phase or a third time span 172 in which an electrical voltage is applied to heating element 148 and heating element 148 is thus switched on, and a fourth phase or a fourth time span 174 in which no electrical voltage is applied to heating element 148 and heating element 148 is thus switched off. Fourth time span 174 follows third time span 172. A sum of third time span 172 and fourth time span 174 is, for example, 500 ms.

In order to carry out a diagnosis of fourth electrical lead 146 that connects second separate terminal P2 to pump cell 140 or to counter-electrode 144, a voltage excitation of second pump cell 140 is carried out by way of control device 122 in order to generate a measured signal at measuring resistor 160. A predetermined electrical voltage 176 is thus applied to second pump cell 140 via common terminal COM. A voltage excitation of second pump cell 140 is carried out by way of excitation signal source 156. The predetermined electrical voltage is raised for a first predetermined time 178, and the predetermined electrical voltage 176 is lowered for a second predetermined time 180. The exact sequence of the modification of the voltage is not relevant. Alternatively, for example, the predetermined electrical voltage can first be lowered or decreased, and then raised. The voltage excitation is carried out in such a way that an integral of the applied electrical voltage for the first predetermined time 178 and second predetermined time 180 has a value of zero. This can be achieved by way of identical voltage amplitudes over time spans of identical length. In the exemplifying embodiment shown, first predetermined time 178 and second predetermined time 180 are of identical length. For example, proceeding from an electrical voltage of 425 mV applied to second pump cell 140, the electrical voltage is raised to 700 mV for a first predetermined time 178 and then lowered to 150 mV for second predetermined time 180. This occurs in a time span in which heating element 148 is switched on, for example in first time span 168 and in third time span 172. This generates a measured signal 182 at measuring resistor 160. Fourth electrically conductive connection 146 is identified as intact if measured signal 182 has a value not equal to zero for first predetermined time 178 and second predetermined time 180, and is identified as defective if measured signal 182 has a value of zero for first predetermined time 178 and second predetermined time 180. In the exemplifying embodiment shown, upon voltage excitation measured signal 182 has an approximately sinusoidal profile 184 (indicated by an arrow) in first time span 168, and has a value of zero in third time span 172, so that the signal at point 186 (indicated by an arrow) does not change. This means that no current is flowing at point 186, which indicates an interruption of fourth electrical lead 146.

In the context of a modification, the voltage excitation encompasses a single modification of the predetermined electrical voltage for a predetermined time. For example, the predetermined electrical voltage is raised for the predetermined time. Fourth electrically conductive lead 146 is identified in this context as intact if measured signal 182 exhibits a value not equal to zero for the predetermined time, and is identified as defective if measured signal 182 exhibits a value of zero for the predetermined time. With sensor 100 in the measurement state, the predetermined electrical voltage is thus briefly raised by the hardware by an amount equal to a specific potential, in the form of a one-time pulse in one direction. What is important here is not the duration of the pulse but the change in voltage. The advantage here is that the time during which measured signal 182 is distorted by the voltage excursion is shorter than in the context of an additional counter-pulse in the form of an offset-free pulse.

FIG. 4 shows a second example of a time course of electrical voltages and a measured signal in sensor 100. Only the differences with respect to the exemplifying embodiment shown in FIG. 3 are described. Identical components or features are labeled with identical reference characters. Time is plotted on X axis 162. Listed from top to bottom, the heating voltage of heating element 148, the electrical voltage U_{P2} applied to second pump cell 140, and the voltage drop U_{P2} at measuring resistor 160 are plotted on Y axis 164. The voltage drop U_{P2} at measuring resistor 160 represents the measured signal. The temperature of sensor element 110 is controlled by pulse width modulation (PWM) of the heating power supply (voltage or current). A curve 166 that represents the heating voltage of heating element 148 correspondingly shows at least a first phase or a first time span 168 in which an electrical voltage is applied to heating element 148 and heating element 148 is thus switched on, and a second phase or a second time span 170 in which no electrical voltage is applied to heating element 148 and heating element 148 is thus switched off. Second time span 170 follows first time span 168. A sum of first time span 168 and second time span 170 is, for example, 500 ms. In the exemplifying embodiment shown, curve 166 furthermore exhibits a third phase or a third time span 172 in which an electrical voltage is applied to heating element 148 and heating element 148 is thus switched on, and a fourth phase or a fourth time span 174 in which no electrical voltage is applied to heating element 148 and heating element 148 is thus switched off. Fourth time span 174 follows third time span 172. A sum of third time span 172 and fourth time span 174 is, for example, 500 ms.

In order to carry out a diagnosis of fourth electrical lead 146 that connects second separate terminal P2 to pump cell 140 or to counter-electrode 144, a current excitation of second pump cell 140 is carried out by way of control device 122 in order to generate a measured signal at measuring resistor 160. For example, a predetermined electrical current is applied to pump cell 140 via common terminal COM. A current excitation of second pump cell 140 is carried out by way of excitation signal source 156. The predetermined electrical current is raised for a first predetermined time 178, and the predetermined electrical current is lowered for a second predetermined time 180. The exact sequence of the modification of the current is not relevant.

Alternatively, for example, the predetermined electrical current can first be lowered or decreased, and then raised. The current excitation is carried out in such a way that an integral of the impressed electrical current has a value of zero for first predetermined time 178 and second predetermined time 180. This can be achieved by way of identical current amplitudes over time spans of identical length. In the exemplifying embodiment shown, first predetermined time 178 and second predetermined time 180 are of identical length. For example, proceeding from an electrical current impressed into second pump cell 140, the electrical current is raised for a first predetermined time 178 and then lowered for second predetermined time 180. This occurs in a time span in which heating element 148 is switched on, for example in first time span 168 and in third time span 172. The current excitation is recognizable from a positive peak 188 and a subsequent negative peak 190 of measured signal 182 at measuring resistor 160. The current excitation generates a voltage excursion in the voltage U_{P2} applied to the second pump cell. Fourth electrically conductive connection 146 is identified as intact if the voltage U_{P2} applied to the second pump cell falls below a threshold value 192 in first predetermined time 178 and second predetermined time 180, and is identified as defective if the voltage U_{P2} applied to the second pump cell exceeds a threshold value 192 in first predetermined time 178 and second predetermined time 180. Threshold value 192 is defined here as a magnitude of the amplitude or absolute value of the change in voltage. In the exemplifying embodiment shown, upon current excitation in first time span 168 the voltage U_{P2} applied to the second pump cell has an approximately sinusoidal profile 194, indicated by an arrow, which falls below threshold value 192, and in third time span 172 has an approximately sinusoidal voltage excursion 196, indicated by an arrow, which exceeds threshold value 192.

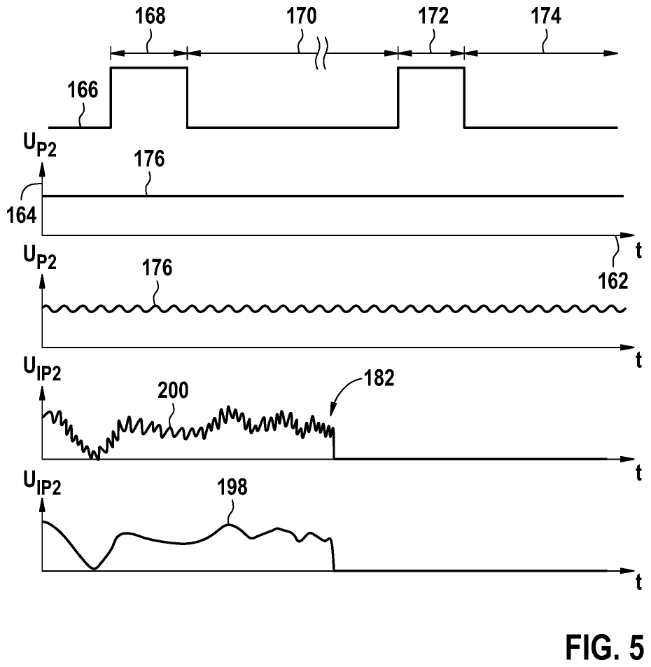

FIG. 5 shows a third example of a time course of electrical voltages and a measured signal in sensor 100. Only the differences with respect to the exemplifying embodiment shown in FIG. 3 are described. Identical components or features are labeled with identical reference characters. Time is plotted on X axis 162. Listed from top to bottom, the heating voltage of heating element 148, the electrical voltage U_{P2} applied to second pump cell 140 without voltage excitation, the electrical voltage U_{P2} applied to second pump cell 140 with voltage excitation, the voltage drop U_{P2} at measuring resistor 160, and the voltage drop U_{P2} at measuring resistor 160 after low-pass filtering, are plotted on Y axis 164. The voltage drop U_{P2} at measuring resistor 160 represents the measured signal. The temperature of sensor element 110 is controlled by pulse width modulation (PWM) of the heating power supply (voltage or current). A curve 166 that represents the heating voltage of heating element 148 correspondingly shows at least a first phase or a first time span 168 in which an electrical voltage is applied to heating element 148 and heating element 148 is thus switched on, and a second phase or a second time span 170 in which no electrical voltage is applied to heating element 148 and heating element 148 is thus switched off. Second time span 170 follows first time span 168. A sum of first time span 168 and second time span 170 is, for example, 500 ms. In the exemplifying embodiment shown, curve 166 furthermore exhibits a third phase or a third time span 172 in which an electrical voltage is applied to heating element 148 and heating element 148 is thus switched on, and a fourth phase or a fourth time span 174 in which no electrical voltage is applied to heating element 148 and heating element 148 is thus switched off. Fourth time span 174 follows third time span 172. A sum of third time span 172 and fourth time span 174 is, for example, 500 ms.

In order to carry out a diagnosis of fourth electrical lead 146 that connects second separate terminal P2 to pump cell 140 or to counter-electrode 144, a voltage excitation of second pump cell 140 is carried out by way of control device 122 in order to generate a measured signal at measuring resistor 160. For example, a predetermined electrical voltage 176 is applied to pump cell 140 via common terminal COM. A voltage excitation of second pump cell 140 is carried out by way of excitation signal source 156. The voltage excitation encompasses a periodic modification of the predetermined electrical voltage. This generates a measured signal 182 at measuring resistor 160, the period length being less than an electrochemical time constant of second pump cell 140. Measured signal 182 at measuring resistor 160 therefore exhibits a superposition of the actual measured signal with the periodic voltage excitation. Measured signal 182 is filtered by way of a low-pass filter (not shown in further detail) of control device 122, and transferred via an interface of the control device to an engine control device. The predetermined electrical voltage is modified at a frequency that is greater than the bandwidth of the low-pass filter. Low-pass filtering of measured signal 182 yields signal 198 from which the periodic voltage excitation has been removed. The measured signal for NOₓ is therefore not distorted. Fourth electrically conductive connection 146 is identified as intact if measured signal 182 before low-pass filtering (i.e., the raw signal) exhibits a periodic change 200, and is identified as defective if measured signal 182 before low-pass filtering exhibits no periodic change 200. A periodic change of this kind is caused by the frequency of the voltage change in the context of an intact electrical connection. In the exemplifying embodiment shown, a periodic change 200 exists in first time span 168, whereas no periodic change 200 exists in third time span 172. This indicates that an interruption of fourth electrical lead 146 exists in third time span 172. The frequency on fourth electrical lead 146 does not obligatorily need to be excited over the entire time span. Application of the higher-frequency voltage change in the region around almost 0 ppm NOₓ is sufficient for electrical diagnosis.

1-16. (canceled)

17. A method for operating a sensor for detecting at least a portion of a measured-gas component having bound oxygen in a measured gas, in an exhaust gas of an internal combustion engine, the sensor including a sensor element having: (i) a first pump cell that has an external pump electrode and an internal pump electrode and adjoins a first cavity that is in communication with the measured gas, (ii) a reference cell that has a Nernst electrode and a reference electrode and adjoins a reference gas space, and (iii) a second pump cell that has a pump electrode and a counter-electrode and adjoins a second cavity, the sensor further including an electronic control device connected to the sensor element, the electronic control device having at least a first separate terminal for the first pump cell and a second separate terminal for the second pump cell, the first pump cell being connected to the electronic control device using an electrically conductive connection to the first separate terminal, the second pump cell being connected to the electronic control device using an electrically conductive connection to the second separate terminal, a measuring resistor being provided in the electrically conductive connection that connects the second pump cell to the second separate terminal, the method comprising:
carrying out a current excitation and/or voltage excitation of the second pump cell using the control device to generate a measured signal at the measuring resistor.

18. The method as recited in claim 17, wherein for the carrying out, a predetermined electrical voltage is applied to the second pump cell, the voltage excitation of the second pump cell being carried out, the voltage excitation encompassing a modification of the predetermined electrical voltage for a predetermined time.

19. The method as recited in claim 18, wherein the predetermined electrical voltage is raised for the predetermined time.

20. The method as recited in claim 18, wherein the electrically conductive connection that connects the second pump cell to the second separate terminal is identified as intact when the measured signal has a value not equal to zero for the predetermined time, and is identified as defective when the measured signal has a value of zero for the predetermined time.

21. The method as recited in claim 17, wherein, for the carrying out, a predetermined electrical voltage is applied to the second pump cell, the voltage excitation of the second pump cell being carried out, the predetermined electrical voltage being raised for a first predetermined time, and the predetermined electrical voltage being lowered for a second predetermined time, an integral of the applied electrical voltage having a value of zero for the first predetermined time and the second predetermined time.

22. The method as recited in claim 21, wherein the electrically conductive connection that connects the second pump cell to the second separate terminal is identified as intact when the measured signal has a value not equal to zero for the first predetermined time and the second predetermined time, and is identified as defective when the measured signal has a value of zero for the first predetermined time and the second predetermined time.

23. The method as recited in claim 17, wherein, for the carrying out, a predetermined electrical current is impressed into the second pump cell, the current excitation of the second pump cell being carried out, the predetermined electrical current being raised for a first predetermined time, and the predetermined electrical current being lowered for a second predetermined time, the first predetermined time and the second predetermined time being identical in length.

24. The method as recited in claim 23, wherein the electrically conductive connection that connects the second pump cell to the second separate terminal is identified as intact when an electrical voltage applied to the second pump cell falls below a threshold value for the first predetermined time and for the second predetermined time, and being identified as defective when an electrical voltage applied to the second pump cell exceeds a threshold value for the first predetermined time and for the second predetermined time.

25. The method as recited in claim 17, wherein, for the carrying out, a predetermined electrical voltage is applied to the second pump cell, and the voltage excitation of the second pump cell is carried out, the voltage excitation encompassing a periodic modification of the predetermined electrical voltage.

26. The method as recited in claim 25, wherein a period length is less than an electrochemical time constant of the second pump cell.

27. The method as recited in claim 25, wherein the measured signal is filtered using a low-pass filter, the predetermined electrical voltage being modified at a frequency that is greater than a bandwidth of the low-pass filter.

28. The method as recited in claim 25, wherein the electrically conductive connection that connects the second pump cell to the second separate terminal is identified as intact when the measured signal exhibits a periodic change, and being identified as defective when the measured signal exhibits no periodic change.

29. A non-transitory electronic storage medium on which is stored a computer program for operating a sensor for detecting at least a portion of a measured-gas component having bound oxygen in a measured gas, in an exhaust gas of an internal combustion engine, the sensor including a sensor element having: (i) a first pump cell that has an external pump electrode and an internal pump electrode and adjoins a first cavity that is in communication with the measured gas, (ii) a reference cell that has a Nernst electrode and a reference electrode and adjoins a reference gas space, and (iii) a second pump cell that has a pump electrode and a counter-electrode and adjoins a second cavity, the sensor further including an electronic control device connected to the sensor element, the electronic control device having at least a first separate terminal for the first pump cell and a second separate terminal for the second pump cell, the first pump cell being connected to the electronic control device using an electrically conductive connection to the first separate terminal, the second pump cell being connected to the electronic control device using an electrically conductive connection to the second separate terminal, a measuring resistor being provided in the electrically conductive connection that connects the second pump cell to the second separate terminal, the computer program, when executed by a computer, causing the computer to perform:
carrying out a current excitation and/or voltage excitation of the second pump cell using the control device to generate a measured signal at the measuring resistor.

30. An electronic control device configured to operate a sensor for detecting at least a portion of a measured-gas component having bound oxygen in a measured gas, in an exhaust gas of an internal combustion engine, the sensor including a sensor element having: (i) a first pump cell that has an external pump electrode and an internal pump electrode and adjoins a first cavity that is in communication with the measured gas, (ii) a reference cell that has a Nernst electrode and a reference electrode and adjoins a reference gas space, and (iii) a second pump cell that has a pump electrode and a counter-electrode and adjoins a second cavity, the electronic control device comprising:
at least a first separate terminal for the first pump cell and a second separate terminal for the second pump cell, the first pump cell being connected to the electronic control device using an electrically conductive connection to the first separate terminal, the second pump cell being connected to the electronic control device using an electrically conductive connection to the second separate terminal, a measuring resistor being provided in the electrically conductive connection that connects the second pump cell to the second separate terminal;
wherein the electronic control device is configured to carry out a current excitation and/or voltage excitation of the second pump cell to generate a measured signal at the measuring resistor.

31. A sensor for detecting at least a portion of a measured-gas component having bound oxygen in a measured gas, in an exhaust gas of an internal combustion engine, comprising:
a sensor element including a first pump cell that has an external pump electrode and an internal pump electrode and adjoins a first cavity that is communication with the measured gas, a reference cell that has a Nernst electrode and a reference electrode and adjoins a reference gas space, and a second pump cell that has a pump electrode and a counter-electrode and adjoins a second cavity; and
an electronic control device connected to the sensor element, the electronic device having at least a first separate terminal for the first pump cell and a second separate terminal for the second pump cell, the first pump cell being connected to the electronic control device using an electrically conductive connection to the first separate terminal, the second pump cell being connected to the electronic control device using an electrically conductive connection to the second separate terminal, a measuring resistor being provided in the electrically conductive connection that connects the second pump cell to the second separate terminal, electronic control device configured to carry out a current excitation and/or voltage excitation of the second pump cell to generate a measured signal at the measuring resistor.
